(19) United States
(12) Patent Application Publication
(10) Pub. No.: US 2003/0206981 A1
(43) Pub. Date: Nov. 6, 2003

(54) Methods of using and compositions comprising cacao extract including dietary fiber

(75) Inventors: Shin-Young Lee, Chuncheon-si (KR); Jung-Suk Lee, Leecheon-si (KR); Ik Boo Kwon, Seoul (KR)

Correspondence Address: PENNIE AND EDMONDS, 1155 AVENUE OF THE AMERICAS, NEW YORK, NY, 100362711

(73) Assignee: Lotte Confectionery Co., Ltd.

(21) Appl. No.: 10/430,015

(22) Filed: May 5, 2003

Related U.S. Application Data
(62) Division of application No. 09/958,873, filed on Dec. 28, 2001.

(30) Foreign Application Priority Data
Apr. 15, 1999 (KR) 1999-13401

(51) Int. Cl. A61K 35/78; A61K 9/14

(52) U.S. Cl. 424/776

(57) ABSTRACT

The present invention relates to cacao extract including dietary fiber, more specifically cacao extract including dietary fiber useful for the treatment of diabetes obtained as a residue of solvent extraction of the cacao bean husk (CBH) conventionally wasted after using only cacao bean (CB), which has the following characteristics:

(a) A high content of dietary fiber is included, and especially, insoluble dietary fiber (IDF) is in excess of soluble dietary fiber (SDF);

(b) The physical and chemical properties of these fibers, such as water holding capacity (WHC), oil binding capacity (OBC), viscosity characteristics and dialysis retardation index of glucose and bile acid; and

(c) It improves the physiological activity of intestinal disease and metabolic disease, for example, blood sugar depression and cholesterol metabolism enhancement.

### CROSS REFERENCE TO RELATED APPLICATION

This application is a division of U.S. patent application Ser. No. 09/958,873 filed on Dec. 28, 2001, the entirety of which is incorporated herein by reference.

### BACKGROUND OF THE INVENTION

1. Field of the Invention

The present invention relates to cacao extract containing dietary fiber, more specifically cacao extract containing dietary fiber useful for the treatment of diabetes obtained as a residue of solvent extraction of the cacao bean husk (CBH) conventionally wasted after using only cacao bean (CB), which has the following characteristics:

a) A high content of dietary fiber is contained, and especially, insoluble dietary fiber (IDF) is in excess of soluble dietary fiber (SDF);

b) The physical and chemical properties of these fibers, such as water holding capacity (WHC), oil binding capacity (OBC), viscosity characteristics and dialysis retardation index of glucose and bile acid; and

c) It improves the physiological activity of intestinal disease and metabolic disease, for example, blood sugar depression and cholesterol metabolism enhancement.

2. Description of the Related Arts

Cacao (Theobroma cacao L.) is a Latin America originated perennial belonging to Byttneriaceae family, which grows as high as 6-8 m. It produces an oval-type pod, in which about 30-40 seeds are embedded by pulp. Cacao comprises a shell (or testa), a nib (or cotyledon) and a germ, and the main ingredient of chocolate is a ground mass made by grinding the nib with high butter content. This mass is called cacao liquor or cacao mass (CM) because it is in a paste phase below the melting point of butter.

While nibs are used as a main ingredient of chocolate, the husks winnowed in the CM production process are by-products of about 15 wt % (about 400,000 M/T worldwide) and wasted entirely. This is called the cacao bean husk (CBH).

While the current chocolate market in Korea is fairly large, the recognition about chocolate is changing recently with the opening of foreign chocolate import. Especially, together with the recent dieting boom or increased consumers' interest in health food related to various intestinal and metabolic diseases, such as diabetes, tooth decay and arteriosclerosis, chocolate is being recognized negatively due to its high sugar content and high-fat calories.

Accordingly, it is required to find out new physiological functions of chocolate as a health food. For the past years, various physiological activities of polyphenols contained in CM, the main component of chocolate, and CBH, processing residues winnowed during the CM producing process, have been found and studied in Japan and other countries. The research of these physiological activities are known in the reference. Especially, since the polyphenol components contained in CB or CM showed a new effect on orterosclerosis, allergic diseases and various heart diseases, they are watched with keen interest about various physiological functions on prevention against cancer, tooth decay prevention, antioxidation and stress prevention. Also in Korea, researches on CM and CBH are being proceeded and there has been a great success in finding out new function related to chocolate by elucidating various physiological activities resulting from these components, for example, function as an inhibitor against enzymes, such as GTase (glycosyltransferase), ACE (angiotesin converting enzyme), Xoase (xanthine oxidase) and tyrosinase.

Another noteworthy component related to the said various physiological functions is dietary fiber which is abundantly contained in cacao. Researches on dietary fiber is presented in the reference. However, since dietary fiber is a structural residue of plant cells indigestible with human digestive enzymes, it has been recognized as having no nutritional value. Although dietary fiber is contained very abundantly in CB or CBH, researches on characteristics and physiological functions are not enough, compared with polyphenols. However, since Trowell's report of 1972 that disease such as obesity, heart diseases, diabetes, intestinal diseases and gallstone are related to insufficient ingestion of dietary fiber, researches on and interest in the physiological functions of dietary fiber is increasing remarkably.

In general, dietary fiber is contained in various food stuffs, and there are many kinds of dietary fiber. Dietary fiber is largely divided into soluble dietary fiber (SDF: indigestible dextrin, pullulan, polydextrose, glucomannan, pectin, enzymolyzed residue of guar gum, rice barn, hemicellulose, agar, etc.) and insoluble dietary fiber (IDF: wheat bran, spirulina, chitosan, corn, apple, soybean, soybean husk, cellulose, ginseng, etc.). Dietary fiber content of various plant food ranges 1-43 wt %.

The physiological activity of dietary fiber is affected by its physical and chemical characteristics, such as water holding capacity (WHC), cation exchange capacity, bile acid binding capacity, fermentability, etc. and varies with composition, content, binding state, cooking condition, source, etc. of dietary fiber. Generally, water absorption capacity of dietary fiber is known to be related to the mechanism lowering digestive capacity, increasing volume and weight of feces, and lowering serum triglyceride. Especially, water absorption capacity of grain fiber is an important decisive factor in increasing the volume of feces. This water absorption capacity is greatly affected by the type, content and particle size of the dietary fiber, and depends mainly on the component, fineness, pH and ionic strength of the dietary fiber.

Researches on various materials are being conducted with regard to diabetes. Among the currently used diabetes treatment methods (diet therapy, kinesiatrics and pharmacotherapy), due to the rising problems of toxicity and tolerance of patients in pharmacotherapy, researches on new drugs obtained from natural products are executed widely.

Among the natural products, although there are a few research reports on dietary fiber from cacao related to blood pressure depression or cholesterol depression, it is not yet put into practical use and continuous researches are not being conducted. Particularly, physical properties and effects on diabetes of various dietary fibers have not been reported yet. Generally, dietary fiber is known to reduce the insulin demand by retarding the glucose absorption, and hence help the diabetes treatment”, Nippon Naibunpi Gakkai ZasshiFolia Endocrinologica Japonica, 68, 623-628 (1992)].

Actually, from a result of clinical researches, intake of dietary fiber has been found to improve glucose retardation and significantly reduce the insulin demand of diabetic patients. In addition, since many complications of diabetes are directly related to hyperglycemia, it is important to reduce after-meal hyperglycemia. It is also reported that prolonged intake of meals containing proper contents of dietary fiber significantly reduces the total serum cholesterol level of type II diabetic patients.

In spite of these researches on dietary fiber, no natural extract with significant effect of diabetes treatment has been found yet. Especially, researches on specific efficacy and effects of cacao extract on various disease are scarce and only at the very early state, and neither diabetes treatment nor marketing of dietary fiber extracted from natural plants has been accomplished.

### SUMMARY OF THE INVENTION

In order to utilize CBH, which are being wasted during cacao mass processing, as a useful source, dietary fiber was separated from the extract residue of CBH and also its characteristics and physiological functions were investigated. As a result, it was found that dietary fiber, useful for diabetes treatment due to the absorption retardation of glucose and bile acid, blood sugar depression and improvement of cholesterol metabolism, is contained in excess in CBH. Accordingly, an object of this invention is to provide a cacao extract containing dietary fiber with excellent effect of diabetes treatment from formerly wasted CBH.

### DETAILED DESCRIPTION OF THE INVENTION

The present invention is characterized by cacao extract obtained from extraction residue of CBH wasted during cacao processing and containing dietary fiber of 43-60 wt %.

The present invention is explained in detail as set forth hereunder.

The present invention relates to cacao extract including dietary fiber solvent-extracted from the formerly wasted CBH, more specifically cacao extract including excess dietary fiber with excellent diabetes treatment compared to cacao bean or other plant sources due to its good physiological activity on the depression of blood sugar and the improvement of blood cholesterol level.

Cacao bean is preheated, ground, selected and winnowed to obtain CBH, and then ethanol is added to CBH. Thereby the cacao extract of the present invention is obtained as residue of extraction.

Extraction residue of CBH is prepared using the L-BTC (Better Taste and Color) method as shown in FIG. 1. The process is explained in detail as set forth hereunder.

Cacao bean is preheated at the temperature of 160-180° C. and under the pressure of 0.5-1 bar, crushed, selected, and winnowed to give CBH. If the pressure is below 0.5 bar, CBH becomes excess in the nib, and if the pressure exceeds 1 bar, the nib yield becomes poor. The purpose of the thermal pretreatment is to make bean soft and easily breakable before grinding it. If the temperature of the thermal pretreatment is below 160° C., CBH is not separated well from the nib, and if the temperature exceeds 180° C., the nib yield becomes poor.

Extraction residue of CBH is obtained by adding ethanol to the said preheated CBH and centrifuging the same. In this process, the polyphenol extract is put aside and used for beverage. Solvents such as ethanol, methanol, butanol, etc. are used for the extraction, and ethanol is the most desirable among them.

The dietary fiber composition is separated using the Prosky method in order to identify the dietary fiber content of the residue. The results of adding enzymes such as temamyl, protease and amyloglucosidase show that the cacao extract of the present invention consists of the mixture of SDF and IDF.

Another substances obtained from the said cacao preparation process of the present invention are winnowed nibs, which are crushed, selected and winnowed, and the reacted nibs, which are obtained by reacting the winnowed nibs for 10-15 mins under the pressure of 1-1.5 bar. Cacao mass (CM), the major constituent of chocolate, is obtained by drying, roasting and grinding the same at the temperature of 130-140° C. and D.R. (discharge rate) of 900-1,100 kg/hr.

Cacao powder, cacao nibs, cacao mass or mixture of these can be mixed additionally to the said extract in the range of 5-15 wt %.

The total dietary fiber content of the defatted CBH, powder, nibs and CM of the present invention is 30-60 wt %, and desirably 43-60 wt %. The dietary fiber content is larger than the conventional content of 1-43 wt % in plant materials, and physiological activities such as absorption retardation of glucose and bile acid are superior.

IDF content of the CBH residue with polyphenol composition removed by the ethanol extraction is higher than that of CBH. Whereas, IDF and SDF contents of the supernatant are very low as 0.2-0.4 wt % and 2-3 wt %, respectively. IDF content of the residue is high because SDF is lost or transferred to the supernatant during the polyphenol extraction process. The CBH extraction residue is high in the carbohydrate content (50-60 wt %) and low in the reduced sugar content. Also, since the residue is macromolecular substance, dietary fiber exists mostly in the residue. The protein content (33.43±0.09 wt %) and crude ash content (18.03±0.67 wt %) are higher in the supernatant. It is because soluble materials such as protein and salts were transferred to the supernatant. Accordingly, this residue is used as high dietary fiber source.

IDF content of CBH is 25-45 wt % and higher than its SDF content of 5-15 wt %. IDF content of cacao bean extract is 29-33 wt % and its SDF content is 3-5 wt %. Although lower than that of CBH, the higher IDF content compared with the SDF content enables cacao bean extract to be used as high dietary fiber source. However, cacao bean is not as economical as CBH. Also, IDF content of CM extract is 25-35 wt % and higher than its SDF content of 1.5-3 wt %. However, as mentioned above, CM alone is applicable for the source of chocolate, but limited and economically unfavorable for high dietary fiber source.

The dietary fiber contained in the cacao extract of the present invention shows high water holding capacity (WHC), oil binding capacity (OBC) and viscosity and excellent retardation effect of glucose and bile acid absorption.

WHC of dietary fiber extracted from CBH, cacao bean (CB) and CM, especially that of CBH extract, of the present invention, is very powerful as 9 times, compared with 8 times of pectin on the market. Whereas, WHC of cacao husk and cacao bean themselves are only about a half of the corresponding extracts.

Oil binding capacity (OBC) of the dietary fiber contained in the cacao extract of the present invention is also excellent. For cacao extract, OBC of the dietary fiber is 35-70% of WHC, and for CM, OBC is 60-70% of WHC. So, efficient adsorption can be attained if used for the processed food containing both water and oil.

Also, since the dietary fiber of the cacao extract of the present invention has viscosity and gel formation ability, it increases the viscosity of the digested material. Hence, it affects the mobility and absorption of the digested material in the digestive duct. Surprisingly, the dietary fiber of the extract residue obtained from the CBH of the present invention shows somewhat higher viscosity than the dietary fiber of CB, and the same has higher viscosity in IDF than in SDF.

The in vitro measurement results of the cacao extract containing dietary fiber of the present invention show very excellent retardation of glucose and bile acid permeation.

The retardation rate of glucose absorption of IDF and SDF of the cacao extract residue of the present invention are 17-20% and 25-30% respectively. It is because SDF's viscosity is higher than that of IDF, since WHC of dietary fiber increases generally with time and gel structure is formed so that physical property is changed and glucose is entrapped. Accordingly, the cacao extract of the present invention is very useful for the high dietary fiber product, because the dietary fiber content is higher than 9-11% which is extracted from a-cellulose currently on the market. However, glucose retardation rate of CBH itself is higher for IDF as 15-30% than that of SDF, being 10-25%. This is because the glucose absorption retardation effect is changed due to the solvent treatment of ethanol extraction, whereas CBH does not go through such treatment. Accordingly, ethanol extraction residue with polyphenol removed is superior to CBH itself. Also, sampling time and sampling place of cacao bean affect the results.

The bile acid absorption retardation of IDF and SDF of the cacao extract residue of the present invention are 30-35% and 60-65%, respectively. It is because SDF's viscosity is higher than that of IDF as explained above. Accordingly, bile acid retardation rate of the cacao extract of the present invention is superior to 5-10% of the conventional SDFs such as citrous pectin, guar gum, etc. Also, the bile acid retardation rates of IDF and SDF of CBH are higher than the conventional, being 35-40% and 55-63% respectively. In addition, the bile acid retardation rates of CB and CM are 30-35%. Therefore, the bile acid retardation effect is very excellent, with SDF's bile acid retardation rates of all extracts except CB extract being almost double that of IDF. It is because SDF's viscosity is higher than that of IDF as explained above.

Physiological activity measurement through in vitro experiment of cacao extract of the present invention shows the reduction of blood insulin level, because sugar absorption at the intestine is retarded due to the reduced mobility of carbohydrate from the stomach to the intestine and inhibited digestion of starch and other saccharides, and blood insulin level is reduced due to the inhibited entry of digested material into the surface of epithelial cells and the consequent inhibited sugar absorption at the intestine. Also, the cacao extract containing dietary fiber of the present invention has powerful effect on prevention and treatment of diabetes and adult disease, because it reduces the neutral lipid level, phospholipid level and cholesterol level of the serum, and regulates the lipid metabolism imbalance.

The following specific examples are intended to be illustrative of the present invention and should not be construed as limiting the scope of the invention as defined by the appended claims.

### EXAMPLES 1-4

Cacao (Theobroma cacao L.) bean of Ghanaian origin, purchased by the L Company, was used for the source material. BTC (Better Taste and Color) method of L Company as shown in FIG. 1, the latest CM processing method of nib roasting process wherein the nib is homogeneously crushed and then roasted, was used. Cacao bean was thermally pretreated at 170° C. and under the pressure of 0.8 bar. After grinding and selecting, the cacao bean was winnowed to obtain CBH. After adding ethanol, the same was extracted, centrifuged at 1,000-3,000 rpm and then fractionated into the supernatant and residue. Winnowed nib obtained during the said winnowing process was treated with nib reaction for 10 min under the pressure of 1.2 bar to give reacted nib. The same was dried, roasted and ground under the condition of 132° C. and D.R. (discharge rate) 1,000 kg/hr to obtain CM, the major source of chocolate.

### EXAMPLE 5

Dietary fiber was extracted and fractionated from the extract residue prepared from the above Example 1 using Prosky method as shown in FIG. 2. The residue was passed through a 30-mesh sieve and skimmed with diethylether for 12 hours using Soxhlet method. After drying at 70° C. in the drying oven, the same was deodored and preserved in the refrigerator after sealing for the use of dietary extract sample. Phosphate buffer solution (0.08M, pH 6, 25 ml) was added to 0.5 g of the prepared residue sample, and the same was treated with temamyl for at 95-10° C. for 30 min and pH was adjusted to 7.5 using NaOH (0.275N, 5 ml), and then treated with protease at 60° C. for 30 min. 5 ml of 0.325M HCl was added to adjust pH to 4-4.6, treated with amyloglucosidase at 60° C. for 30 min, washed with distilled water and filtered to obtain dietary fiber filtrate and residue. Ethanol (95%) was added to the dietary fiber filtrate at 60° C. and the same was filtered again, washed with ethanol (78%, 95%) and acetone, dried and weighed to obtain IDF. The same procedure was applied for CB, winnowed nib, reacted nib and CM to obtain dietary fiber.

### EXAMPLE 6

The yield of Example 1 was 12.63±1.38 wt % for CBH, 86.77±1.52 wt % for winnowed nib, 95.95±0.49 wt % for reacted nib and 84.18±0.23 wt % for CM. Extract was prepared by adding solvent and mixing each, the solvent was removed and the same was dried. The dietary fiber was fractionated and extracted as in the Example 2 for the identification of the dietary fiber content.

### COMPARATIVE EXAMPLES 1-3

Commercially available chicory, aloe vera and yacon were used for the comparative example. Dietary fiber was fractionated and extracted using the Prosky method of the Example 5.

### EXPERIMENTAL EXAMPLE 1

### Dietary Fiber Content and Composition of the Cacao Extract

a) IDF Content

IDF content of the prepared cacao extract was determined using the Prosky method. Two 0.5 g portions of the each extract were treated with enzymatic hydrolysis with total dietary analysis method of AOAC (Association of Official Analytical Chemists) method. The solution (enzyme digest) was suction-filtered to B2 crucible and 500 ml suction flask. The same was washed with acetone (200 ml, 2 times) and suction filtered again. One of the two B2 crucibles containing dietary fiber was incinerated to obtain the ash content (A), and the other was used to obtain the protein content (P). The ash content was measured with direct incineration at 550° C. and the protein content was measured with Kjeldahl (Kjeltec Auto 1035 Sampler System, Tecator, Sweden) method. The same procedure was performed for the blank. The measurement obtained from the blank (B) was subtracted from the dry weight, and IDF content was calculated using the following Equation 1. The results are shown in Table 1.

$$IDF ( \% ) = \frac{\frac{{R}_{1} + {R}_{2}}{2} - P - A - B}{\frac{{S}_{1} + {S}_{2}}{2}} \times 100 \\ Blank ( mg ) = \frac{{R}_{1} + {R}_{2}}{2} - P - A \\ {R}_{1} , {R}_{2} : Weight of residues ; \\ {S}_{1} , {S}_{2} : Weight of samples ; \\ P : Protein content of IDF ; \\ A : Ash content of IDF ; and \\ B : Blank \qquad Equation 1$$

b) SDF Content

SDF content of the prepared cacao extract was determined using the Prosky method as in Experiment 1. Distilled water was added to the filtrate of the 500 ml suction flask saved during the IDF analysis to 100 ml. After pouring the same to a 600 ml beaker, ethanol (95 wt %, 400 ml) preheated to 60° C. was added. The same was left stationary for 60 min at room temperature, and SDF was aggregated and precipitated. This solution was suction filtered to 2 glass crucibles (1 G3 crucible), and was washed two times with 20 ml of ethanol (95 wt %) and two times with 20 ml of acetone. And then, as in Experiment 1, ash content (A) was determined from one of the two 1G3 crucibles and protein content (P) was determined from the other. The same procedure was performed for blank and SDF content was calculated using the following Equation 2.

$$SDF ( \% ) = \frac{\frac{{R}_{1} + {R}_{2}}{2} - P - A - B}{\frac{{S}_{1} + {S}_{2}}{2}} \times 100 \\ Blank ( mg ) = \frac{{R}_{1} + {R}_{2}}{2} - P - A \\ {R}_{1} , {R}_{2} : Weight of residues ; \\ {S}_{1} , {S}_{2} : Weight of samples ; \\ P : Protein content of SDF ; \\ A : Ash content of SDF ; and \\ B : Blank \qquad Equation 2$$

c) TDF (Total Dietary Fiber) Content

TDF content of the prepared cacao extract was calculated by adding IDF to

SDF (TDF, wt %=IDF+SDF). The results are also shown in Table 1.

TABLE 1

| | | | IDF (wt %) | SDF (wt %) | TDF (wt %) |
|---|---|---|---|---|---|
| Examples | 1 | Residue | 47 | 8 | 55 |
| | 2 | CBH | 42.2 | 11.9 | 54.1 |
| | 3 | CB | 29.8 | 3.55 | 33.35 |
| | 4 | CM | 30.3 | 1.97 | 32.27 |
| Comparative | 1 | Chicory | 10.28 | 6.15 | 16.43 |
| Examples | 2 | Aloe vera | 15.43 | 14.35 | 29.78 |
| | 3 | Yacon | 6.49 | 3.1 | 9.58 |

d) Composition of IDF

Contents of NDF (neutral detergent fiber) containing cellulose, hemicellulose and lignin, and ADF (acid detergent fiber) containing cellulose and lignin were determined.

Cellulose content was obtained by the difference of ADF content and lignin content, hemicellulose content was obtained by the difference of NDF content and ADF content, and pectin content was obtained by the difference between TDF content and NDF content, as the following Equation 3.

Cellulose (wt %)=ADF−lignin Equation 3

Hemicellulose (wt %)=NDF−ADF

Pectin (wt %)=TDF−NDF

NDF content was determined by the following procedure. Partially modified van Soest and Wine's method was used for quantification. 1.7 g of α-amylase (Type VI-B, A-3176, from porcine pancreas) and 100 ml of phosphate buffer solution (0.1M, pH 7.0) were taken to a 200 ml flask, and dissolved under agitation for 15 min. The same was centrifuged at 4° C. under 1,500×g for 10 min. The supernatant was filtrated to 1G3 crucible at 4° C., washed with α-amylase solution and preserved in a refrigerator. 1 g of cacao extract sample was weighed to a 50 ml flask and 30 ml of the prepared α-amylase solution was added to the same. After incubation for 12 hr in a constant-temperature water bath, the same was transferred to a reflux flask. 100 ml of neutral detergent solution (a solution prepared by dissolving 30 g of sodium laurylsulphate, 18.61 g of disodium EDTA, 6.81 g of sodium borate decahydrate and 4.56 g of hydrogen phosphate (anhydride) sequentially in 1 l of distilled water, adding 10 ml of 2-ethoxyethanlol and adjusting pH to 6.9-7.1 with H₃PO₄ solution at room temperature) and 2 ml of decahydronaphthalene were added sequentially. After dissolving 0.5 g of sodium sulphite into the solution, the same was heated for 60 min in a boiling constant-temperature water bath with a countercurrent cooler, and the 1G3 crucible was incinerated for 1 hr in a Maffle's furnace at 525° C., was dried for 3 hr in a drying oven and weighed. Heated solution was suction filtered to this 1G3 crucible, and washed two times with hot distilled water. 1 G3 crucible containing NDF was dried for 12 hr in a drying oven, and left to be cooled. After incarnating for 3 hr at 525° C., the same was weighed. The same procedure was performed for the blank, and NDF content was determined by the following Equation 4.

$$NDF ( \% ) = \frac{{W}_{1} - {W}_{2} - B}{S} \times 100 \\ S : Weight of sample \\ {W}_{1} : Weight after drying \\ {W}_{2} : Weight after incineration \\ B : Weight of blank \qquad Equation 4$$

ADF content was measured using AOAC method. After weighing 1 g of the sample to a 500 ml reflux flask, 100 ml of acid detergent solution (20 g of acetyltrimethyl ammonium bromide dissolved in 1 l of 1N sulfuric acid) was added and the same was heated for 60 min in a boiling constant-temperature water-bath with a countercurrent cooler. After the 1G3 crucible was incinerated for 1 hr at 525° C., the same was dried in a drying oven for 3 hr at 105° C. and weighed after cooling. The heated solution was suction-filtered to this 1G3 crucible and washed thoroughly with about 100 ml of hot distilled water and detergent, and then rinsed two times with 20 ml of acetone. 1G3 crucible containing ADF was dried in a drying oven for 12 hr at 105° C., and weighed after cooling. ADF content was calculated using the following Equation 5.

$$ADF ( \% ) = \frac{{W}_{1} - {W}_{2}}{S} \times 100 \\ S : Weight of sample ( g ) \\ {W}_{1} : Weight of crucible ( g ) \\ {W}_{2} : Weight after drying ( g ) \qquad Equation 5$$

Lignin content was calculated using AOAC method. The 1G3 crucible containing ADF was put into a 50 ml beaker, and 72 wt % of sulfuric acid (15° C.) was added to the crucible to cover the surface of the residue. The solution was homogenized by stirring with a glass rod and 72 wt % of sulfuric acid was poured to about ⅔ of the crucible. The same was kept stationary for 3 hr while agitating at 20-23° C. every 1 hr. The resultant solution was suction-filtered, and acid was thoroughly removed by washing with about 100 ml of hot distilled water. The crucible containing lignin was dried in a drying oven for 12 hr at 105° C. and weighed after cooling. The same was weighed again after incinerating for 3 hr at 500° C. The same procedure was performed for the blank, and the lignin content was calculated by subtracting this weight from the weight after drying.

$$Lignin ( \% ) = \frac{{W}_{1} - {W}_{2} - B}{S} \times 100 \\ S : Weight of sample ( g ) \\ {W}_{1} : Dry weight after the treatment of 72 wt \% of sulfuric acid \\ {W}_{2} : Weight after incineration \\ B : Weight of blank \qquad Equation 6$$

ADF content and NDF content were calculated from the above equations and the results are shown in Table 2.

TABLE 2

| Sample | ADF (wt %) | NDF (wt %) |
|---|---|---|
| Residue | 39.38 ± 0.09 | 43.95 ± 0.05 |
| CBH | 36.30 ± 0.28¹ | 41.65 ± 2.19 |
| CB | 28.51 ± 2.76 | 30.51 ± 0.14 |
| Winnowed nib | 27.60 ± 0.42 | 30.90 ± 1.84 |
| Reacted nib | 28.80 ± 1.27 | 30.95 ± 0.49 |
| CM | 26.50 ± 0.71 | 30.25 ± 1.06 |

¹Mean ± S.D.

For CBH, the content of cellulose, hemicellulose, lignin and pectin was 18.55 wt %, 5.35 wt %, 17.75 wt % and 12.45 wt %, respectively. For the extraction residue of CBH, cellulose content, hemicellulose content, lignin content and pectin content was 20.05 wt %, 4.57 wt %, 19.33 wt % and 9.66 wt %, respectively. So, all the contents increased in a degree compared with CBH due to the increased IDF content. For CB, cellulose content, hemicellulose content, lignin content and pectin content was 9.26 wt %, 2 wt %, 19.25 wt % and 2.84 wt % respectively. For winnowed nib, cellulose content, hemicellulose content, lignin content and pectin content was 7.27 wt %, 3.3 wt %, 20.33 wt % and 2.72 wt % respectively. For reacted nib, cellulose content, hemicellulose content, lignin content and pectin content was 6.9 wt %, 2.25 wt %, 21.8 wt % and 2.02 wt % respectively. For CM, cellulose content, hemicellulose content, lignin content and pectin content was 6.05 wt %, 3.75 wt %, 20.45 wt % and 0.53 wt %, respectively. The results show that the lignin content of the extract of the present invention is much greater than the conventional grain's 1.98 wt % and vegetable's 0.29 wt %.

### EXPERIMENTAL EXAMPLE 2

### Physical Properties of Dietary Fiber of Cacao

a) Water Holding Capacity (WHC)

Following the method of Sosulski and Cadden's, 1 g of dietary fiber sample extracted from CBH, CB and CM was put into a 50 ml centrifuge cell, and 20 ml of distilled water was added. After 10 min, the same was stirred with a glass rod three times for 30 sec every 10 min, and the residue was weighed after removing the supernatant after centrifugation for 25 min at 10,000 rpm. WHC of extract from CBH, CB and CM was 9.12, 9.20 and 3.52 g water retained/g solid, respectively.

b) Oil Binding Capacity (OBC)

Following the method of Lin, et al., 1 g of dietary fiber sample extracted from CBH, CB and CM was put into a centrifuge cell, and 5 ml of soybean oil (Haipyo Ltd.) was added. After agitating for 30 sec every 5 min during a 30-min period, the same was centrifuged for 25 min at 10,000 rpm, and the residue was weighed after removing the supernatant. OBC of extract from CBH, CB and CM was 3.30, 3.72 and 2.34 g oil retained/g solid, respectively, which falls in the range of 36.2-66.5% of WHC.

c) Viscosity

Each dietary fiber of CBH, CB and CM extracts prepared from the Examples 1-3 was suspended, either alone or by mixing 0.1-0.5 wt % of stabilizer (pectin) and suspending in distilled water, to 1-3 wt % suspension. After heating at 60° C. and cooling to 30° C., the viscosity was measured with a rotating viscometer (Brookfield DV-II+, Brookfield Eng. Labs Inc.) or a capillary viscometer (Cannon-Fenske, Cannon Instrument Co.) The viscosity of SDF of CBH and CB (1% (w/v)) was 1.363 and 1.339 mPa·s respectively. When the thickener (0.1% of pectin and guar gum) was added, the viscosity was 1.5 times (1.902-2.907 mPa·s) and two times (3.803-3.858 mPa·s), respectively. When 1% of IDF was added to the thickener solution, the viscosity of the suspension was 1.57 and 3.16 mPa·s for 0.1% of pectin and guar gum, respectively.

The viscosity of dietary fiber of CBH and CB (1% (w/v)) in 0.1% of pectin solution was 4.47 and 3.97 mPa·s, respectively, and the viscosity of dietary fiber of CBH and CB (1% (w/v)) in 0.1% of guar gum solution was 8.22 and 7.48 mPa·s respectively. The apparent viscosity of 2% CBH and CB in 0.1% guar gum solution at 50 rpm was 21.76 and 17.01 mPa·s, respectively.

### EXPERIMENTAL EXAMPLE 3

### In Vitro Glucose Retardation Effect

Semipermeable membrane permeation method of Adiotomre et al., using the principle that dietary fiber retards the permeation of glucose and bile acid through dialyzing membrane, was used. Since the fact that free glucose passes through dialyzing membrane freely but glucose adsorbed to macromolecular substance cannot pass through it, glucose concentration of dialyzed solution was analyzed and determined. Dialyzing membrane (Sigma D7884: M.W. cut-off <1200) with 3.2 Cm² of area and 10 Cm of length was submerged in 0.1 wt % of sodium azide solution overnight and one end of the dialyzing membrane was tied tightly with cotton thread. After inserting 0.2 g of dietary fiber sample of cacao extract, 6 ml of 0.1 wt % sodium azide solution wherein 36 mg of glucose is dissolved was added. After tightly tying the other end of the dialyzing membrane, the same was put into a 150 ml container and hydrated for 14 hr. The same procedure without dietary fiber sample was performed for the control group. After hydration was terminated, 100 ml of 0.1 wt % sodium azide solution was added to the container and maintained at 37° C. Permeation experiment was performed for 24 hr at 100 rpm in a constant-temperature water-bath. 1 ml of dialyzed solution was taken for constant intervals (30 min for 24 hr) for measurement of glucose content. The retardation effect was calculated by the following equation.

$$Glucose Retardation Index ( \% ) = 100 - \frac{Total glucose diffused from the sack containing dietary fiber \times 100}{Total glucose diffused from the sack not containing dietary fiber} \qquad Equation 7$$

The glucose content was measured using ABTS method. 5 ml of ABTS indicator (a solution prepared by dissolving 60 mg of glucose oxidase and 6 mg of peroxidase in 250 ml of 0.12M phosphate buffer solution) was added to dietary fiber sample of cacao extract dissolved in 0.1 wt % of sodium azide solution and 1 ml of standard glucose solution. Absorbance was measured at 450 nm after putting the same for 30-40 min at room temperature. Glucose change with time and retardation index were calculated, and the results are shown in the following Table 3.

TABLE 3

| | | Dialysis for 30 min | | Dialysis for 60 min | | Dialysis for 120 min | |
|---|---|---|---|---|---|---|---|
| | | Glucose in | | Glucose in | | Glucose in | |
| | | dialyzate | GDRI³ | dialyzate | GDRI | dialyzate | GDRI |
| Sample | | (mg/dl) | (%) | (mg/dl) | (%) | (mg/dl) | (%) |
| Control | | 9.95 ± 1.20¹ | 0 | 14 ± 0.98 | 0 | 21.95 ± 0.35 | 0 |
| IDF | Residue | 8.23 ± 0.48 | 17.29 | 11.41 ± 0.86 | 18.50 | 18.05 ± 0.24 | 17.77 |
| | CBH | 7.10 ± 0.47 | 28.64 | 10.64 ± 0.80 | 24.00 | 18.26 ± 1.26 | 16.81 |
| | CB | 8.60 ± 0.84 | 13.57 | 10.43 ± 0.38 | 25.50 | 17.69 ± 0.10 | 19.41 |
| | CM | 8.07 ± 0.21 | 18.89 | 10.58 ± 0.29 | 24.44 | 18.22 ± 0.14 | 16.99 |
| SDF | Residue | 7.33 ± 0.25 | 26.33 | 10.03 ± 0.35 | 28.36 | 17.40 ± 0.87 | 20.73 |
| | CBH | 7.73 ± 0.33 | 22.31 | 11.62 ± 0.19 | 17.00 | 19.78 ± 1.03 | 9.89 |
| | CB | 7.60 ± 0.95 | 23.62 | 10.90 ± 0.64 | 22.14 | 14.20 ± 0.32 | 35.31 |
| | CM | 8.16 ± 0.10 | 17.99 | 11.99 ± 0.53 | 14.36 | 19.67 ± 0.98 | 10.39 |

¹Mean ± S.D.
³Glucose dialysis retardation index

### EXPERIMENTAL EXAMPLE 4

### In Vitro Bile Acid Retardation Effect

The same method as in Experimental example 3 was used since only the free bile acid passes through dialyzing membrane. Namely, after inserting 0.2 g of dietary fiber sample of cacao extract, 6 ml of the solution prepared by dissolving 15 mmol of taurocholic acid (Sigma T-4009) in 1 l of 0.05M phosphate buffer solution (pH 7.0) prepared from 0.1 wt % sodium azide solution was added in the dialyzing membrane. After tightly tying the end of the dialyzing membrane, the same was put into a 150 ml container and hydrated for 14 hr. The same procedure without dietary fiber sample was performed for the control group. After hydration was terminated, 100 ml of 0.05 M phosphate buffer solution prepared from 0.1 wt % sodium azide solution was added to the container and maintained at 37° C. Permeation experiment was performed for 72 hr at 100 rpm in a constant-temperature water-bath. 1 ml of dialyzed solution was taken for constant intervals for measurement of bile acid content. The bile acid retardation effect was calculated by the following equation.

$$Bile Acid Retardation Index ( \% ) = 100 - \frac{total bile acid diffused from the sac containing dietary fiber}{total bile acid diffused from the sac not containing dietary fiber} \times 100 \qquad Equation 8$$

The bile acid content was measured using method of Boyd, et al. 5 Ml of The bile acid content was measured using method of Boyd, et al. 5 ml of sulfuric acid solution was added to 1 ml of the bile acid (Sigma B-8756) solution, and 1 ml of 0.25 wt % furfural solution was added after 5 min. The same was put for 60 min, and the absorbance was measured at 510 nm when the pink color was developed at its best. Change in the amount of taurocholic acid across the dialyzing membrane was measured. The bile acid retardation rate (%) was calculated and the result is shown in the following Table 4.

TABLE 4

| | | Dialysis for 1 hr | | Dialysis for 2 hr | | Dialysis for 4 hr | |
|---|---|---|---|---|---|---|---|
| | | Bile acid in | | Bile acid in | | Bile acid in | |
| | | dialyzate | BDRI³ | dialyzate | BDRI | dialyzate | BDRI |
| Sample | | (mmol/l) | (%) | (mmol/l) | (%) | (mmol/l) | (%) |
| Control | | 0.126 ± 0.018¹ | 0 | 0.26 ± 0.016 | 0 | 0.352 ± 0.019 | 0 |
| IDF | Residue | 0.071 ± 0.02 | 31.29 | 0.171 ± 0.015 | 32.43 | 0.214 ± 0.012 | 44.24 |
| | CBH | 0.078 ± 0.013 | 38.10 | 0.168 ± 0.020 | 35.38 | 0.296 ± 0.022 | 15.91 |
| | CB | 0.112 ± 0.002 | 11.11 | 0.170 ± 0.01 | 34.62 | 0.236 ± 0.004 | 32.95 |
| | CM | 0.084 ± 0.028 | 33.33 | 0.172 ± 0.008 | 33.85 | 0.198 ± 0.024 | 43.75 |
| SDF | Residue | 0.725 ± 0.018 | 50.26 | 0.121 ± 0.003 | 62.43 | 0.134 ± 0.016 | 66.31 |
| | CBH | 0.064 ± 0.012 | 49.20 | 0.104 ± 0.024 | 60.00 | 0.12 ± 0.014 | 65.91 |
| | CB | 0.11 ± 0.006 | 12.70 | 0.184 ± 0.003 | 29.23 | 0.252 ± 0.028 | 28.41 |
| | CM | 0.066 ± 0.010 | 47.62 | 0.112 ± 0.008 | 56.92 | 0.15 ± 0.01 | 57.39 |

¹Mean ± S.D.
³Bile acid dialysis retardation rate

### EXPERIMENTAL EXAMPLE 5

### Diabetes Treatment Effect of Cacao Extract Containing Dietary Fiber

a) Diet & Water Intake and Change of Weight & Fecal Weight Using Experimental Animal

Animal experiment was performed using rats which were induced to diabetes with Streptozotocin (STZ, Sigma S-0130) in order to investigate the physiological effects such as blood glucose level and improvement of lipid metabolism. Experimental animals used were 5-week-old male S.D. (Sprague Dawley), bought from the Korea Animal Experiment Center Ltd. The standard dietary composition (wt %) for the animal experiment is shown in Table 5, and water intake, weight and fecal weight are shown in Table 6.

TABLE 5

| Ingredient | Control | CBH-R | CBH-S | CM | CM-NDF⁴ |
|---|---|---|---|---|---|
| Casein | 20.0 | 19.2 | 18.3 | 18.5 | 20.0 |
| DL-Methionine | 0.3 | 0.3 | 0.3 | 0.3 | 0.3 |
| Sucrose | 50.0 | 50.0 | 50.0 | 50.0 | 50.0 |
| Cornstarch | 15.0 | 11.8 | 11.7 | 11.6 | 15.0 |
| Cellulose | 5.0 | 5.0 | 5.0 | 5.0 | 0 |
| Corn oil | 5.0 | 3.9 | 4.9 | 48.7 | 5.0 |
| Choline bitartrate | 0.2 | 0.2 | 0.2 | 0.2 | 0.2 |
| Mineral mixture⁵ | 3.5 | 3.5 | 3.5 | 3.5 | 3.5 |
| Vitamin mixture⁵ | 1.0 | 1.0 | 1.0 | 1.0 | 1.0 |
| CBH-R | — | 5.0 | — | — | — |
| CBH-S | — | — | 5.0 | — | — |
| CM | — | — | — | 5.0 | — |
| CM-NDF⁴ | — | — | — | — | 5.0 |

⁴Neutral dietary fiber isolated from cacao mass
⁵AIN-76

TABLE 6

| | Initial body | | | Fecal | Weight |
|---|---|---|---|---|---|
| | weight | Diet intake | Water intake | weight | change (g/2 |
| Group¹ | (g) | (g/day) | (g/day) | (g/day) | weeks) |
| Control | 183.8 ± 17.3¹ | 25.6 ± 2.5 | 208.3 ± 7.7 | 2.1 ± 0.3 | −13.7 |
| CBH-R | 184.3 ± 19.1 | 24.6 ± 3.8 | 187.3 ± 7.7 | 3.1 ± 0.3 | −4.3 |
| CBH-S | 189.3 ± 8.9 | 25.9 ± 2.1 | 190.9 ± 8.3 | 2.5 ± 0.3 | −29.0 |
| CM | 186.2 ± 15.5 | 28.0 ± 1.5 | 168.3 ± 20.2 | 4.8 ± 1.3 | +3.8 |
| CM-NDF³ | 183.2 ± 12.2 | 23.9 ± 3.8 | 194.1 ± 31.1 | 1.5 ± 0.4 | −19.1 |

¹Mean ± S.D.
³Neutral dietary fiber isolated from cacao mass

Experimental animals were raised under the condition of temperature of 20-22° C., humidity of 50±10% and 12 hr of bright and dim period. They were adapted to solid feed for 1 week in the stainless steel cage, and were induced to diabetes. Diabetes induction was performed by intraperitoneally injecting Streptozotocin, which was dissolved in citrate buffer solution (pH 4.5) with the concentration of 50 mg/kg, and fasting the same for 12 hr. After taking out blood from the tails, only rats with blood glucose level higher than 180 mg/dl were used as diabetic rats. For control group, 0.01M of citrate buffer solution was injected by the same method as the diabetic group. After 2 weeks of adaptation, the experimental animals were classified as normal standard diet group, diabetic standard diet group, CBH-R group, CBH-S group, CM group and CM-NDF group. The animals were fed for additional 2 weeks with experimental diet, and 8 rats with similar weight were classified in the same group. Distilled water was provided and the diet was given freely. Diet intake, fecal weight and water intake were measured every day, and the weight was measured every two days.

b) Measurement of Blood Glucose Level and Total Glycolated Hemoglobin

After fasting the experimental animals for 12 hr, the same were anesthetized lightly with ethyl ether, and the whole blood was taken from the heart for use as sample of glycolated hemoglobin analysis. The whole blood was passed through a micro haematocrit tube (GRAF Cat. No. 5.530-06), and centrifuged at 3,000 rpm for 10 min to isolate the blood serum. The same was used as sample for the analysis of blood sugar and lipid. The liver was resected and then treated with saline. After removing water from the same with a filter paper (Whatman No. 2), the weight was measured. The samples were preserved in a refrigerator at −70° C. before analysis. The measured total glycolated hemoglobin is shown in Table 7.

TABLE 7

| | Glucose (mg/dl) | | |
|---|---|---|---|
| Group | Week 1 | Week 2 | GHb (%) |
| Control | 390.3 ± 67.6¹ | 581.2 ± 43.2^{a} | 23.4 ± 11.4^{a} |
| CBH-R | 426.8 ± 23.6 | 537.0 ± 22.4^{a} | 16.4 ± 6.7^{a} |
| CBH-S | 224.6 ± 121.5 | 537.5 ± 59.9^{a} | 17.0 ± 2.4^{a} |
| CM | 169.3 ± 98.5 | 430.2 ± 87.6^{b} | 12.5 ± 0.4^{a} |
| CM-NDF³ | 279.7 ± 152.9 | 416.3 ± 66.1^{b} | 15.9 ± 5.4^{a} |

¹Mean ± S.D.
³Neutral dietary fiber isolated from cacao mass
Mean with the same lettered superscripts in a column are not significantly different (p < 0.05).

The blood sugar was measured at 500 nm by GOD (glucose-oxidase) method using blood sugar measurement kit (AM 201-K) of Asan Pharmaceuticals after isolating the blood serum.

Concentration of neutral lipid (triglyceride, TG) and phospholipid

The concentration of neutral lipid and phospholipid was measured at 550 nm and 500 nm by enzymatic colorimetric method using the kit of Asan Pharmaceuticals (AM 1575-K) and latron kit (PL-E(OM)) after isolating the blood serum.

The effect of dietary fiber of cacao extract on the concentration of neutral lipid (triglyceride, TG) and phospholipid in the blood serum of diabetic animals are shown in Table 8.

TABLE 8

Measuring items

| Group | Neutral lipid | Phospholipid |
|---|---|---|
| Control | 109.04 ± 42.48^{a,1} | 140.17 ± 3.93 |
| CBH-R | 110.00 ± 46.49^{a} | 145.41 ± 1.31 |
| CBH-S | 92.30 ± 45.68^{a} | 204.36 ± 23.75 |
| CM | 81.53 ± 9.79^{a} | 134.27 ± 15.74 |
| CM-NDF³ | 98.71 ± 44.65^{a} | 125.76 ± 4.72 |

¹Mean ± S.D.
³Neutral dietary fiber isolated from cacao mass
Mean with the same lettered superscripts in a column are not significantly different (p < 0.05).

c) Cholesterol Content

The total cholesterol and HDL (high-density lipoprotein)-cholesterol level were measured at 500 nm by enzymatic calorimetric method using the kit of Asan Pharmaceuticals (Cholesterol-enzyme and AM 203-K) after isolating the blood serum. LDL (low-density lipoprotein)-cholesterol was calculated with the following Friedewald's equation. AI (atherogenic index) was also calculated as in Equation 9.

$$\begin{matrix}\begin{matrix}LDL cholesterol ( mg \text{/} dl ) = \\ Total cholesterol - ( Neutral lipid / 5 + HDL cholesterol ) \end{matrix} \\ AI = \frac{Total cholesterol - HDL cholesterol}{HDL cholesterol}\end{matrix} \qquad Equation 9$$

The effect of dietary fiber of cacao extract on the total cholesterol, HDL-cholesterol, LDL-cholesterol and atherogenic index (AI) in the blood into the experimental animals were measured two weeks after the injection of STZ. The result is shown in FIG. 9. SAS (Statistical Analysis System) program was used to investigate the statistical significance of the measurement data obtained from the animal experiments. The significance of each test group was tested using Duncan's multiple test with p<0.05.

TABLE 9

| | Total cholesterol | HDL-cholesterol | LDL-cholesterol | Atherogenic |
|---|---|---|---|---|
| Group | (mg/dl) | (mg/dl) | (mg/dl)⁴ | index (AI)⁵ |
| Control | 100.24 ± 2.97¹ | 32.61 ± 2.06 | 45.82 ± 7.57 | 2.07 ± 0.08 |
| CBH-R | 95.43 ± 2.58 | 43.8 ± 5.29 | 29.62 ± 2.89 | 1.18 ± 0.40 |
| CBH-S | 125.57 ± 9.28 | 56.08 ± 5.32 | 51.03 ± 1.93 | 1.24 ± 0.51 |
| CM | 102.71 ± 3.03 | 40.39 ± 1.74 | 46.69 ± 5.31 | 1.54 ± 0.37 |
| CM-NDF³ | 87.85 ± 8.42 | 40.49 ± 3.82 | 28.66 ± 2.69 | 1.17 ± 0.34 |

¹Mean ± S.D.
³Neutral Dietary Fiber isolated from cacao mass
⁴Total cholesterol - (Neutral lipid/5 + HDL cholesterol)
⁵(Total cholesterol - HDL cholesterol)/HDL cholesterol
Mean with the same lettered superscripts in a colunm are not significantly different (p < 0.05).

As described above, because the cacao extract of the present invention uses CBH which has been wasted in the conventional CM processing, it provides the advantage of cost reduction and is environmental-friendly. Especially, the extract residue of CBH of the present invention contains a lot of dietary fiber, and the dietary fiber is rich in lignin. Therefore, it provides a probability of cacao extract product useful for the treatment of diabetes due to the excellent glucose and retardation effect of bile acid absorption, blood sugar depression and improvement of lipid metabolism.

What is claimed is:

1. A method of treating or preventing diabetes, which comprises administering to a patient in need of such treatment or prevention a therapeutically or prophylactically effective amount of a cacao extract comprising 43-60 wt % of dietary fiber, which fiber is obtained as extract residue of cacao bean husk waste formed during a cacao bean processing, wherein insoluble dietary fiber is in the range of 25-45 wt % and soluble dietary fiber is in the range of 5-15 wt %.

2. A method of treating or preventing a disease ameliorated by the reduction of level of cholesterol, lipid or phospholipid in a patient which comprises administering to a patient in need of such treatment or prevention a therapeutically or prophylactically effective amount of a cacao extract comprising 43-60 wt % of dietary fiber, which fiber is obtained as extract residue of cacao bean husk waste formed during a cacao bean processing, wherein insoluble dietary fiber is in the range of 25-45 wt % and soluble dietary fiber is in the range of 5-15 wt %.

3. The method of claim 2, wherein the disease is hyperlipidemia, arteriosclerosis, angina pectoris, stroke or fatty river.

4. The method of claim 1, 2 or 3, wherein the cacao extract is administered in the form of a pharmaceutical composition.

5. The method of claim 1, 2 or 3, wherein the cacao extract is administered in the form of a food composition.

6. A pharmaceutical composition suitable for treating or preventing diabetes or a disease ameliorated by the reduction of level of cholesterol, lipid or phospholipid, which comprises a cacao extract comprising 43-60 wt % of dietary fiber, which is obtained as extract residue of cacao bean husk wasted during a cacao bean processing wherein insoluble dietary fiber is in the range of 25-45 wt % and soluble dietary fiber is in the range of 5-15 wt %.

7. The pharmaceutical composition of claim 6 further comprising a pharmaceutically acceptable carrier, excipient or diluent.

8. A food composition suitable for treating or preventing diabetes or a disease ameliorated by the reduction of level of cholesterol, lipid or phospholipid, which comprises a cacao extract comprising 43-60 wt % of dietary fiber, which is obtained as extract residue of cacao bean husk wasted during a cacao bean processing wherein insoluble dietary fiber is in the range of 25-45 wt % and soluble dietary fiber is in the range of 5-15 wt %.
